(19) United States
(12) Patent Application Publication
(10) Pub. No.: US 2021/0087571 A1
(43) Pub. Date: Mar. 25, 2021

(54) ACETYL-COA-DERIVED BIOSYNTHESIS

(71) Applicant: William Marsh Rice University, Houston, TX (US)

(72) Inventors: Ka-Yiu SAN, Houston, TX (US); Zhilin LI, Houston, TX (US)

(21) Appl. No.: 16/970,852

(22) PCT Filed: Feb. 26, 2019
(86) PCT No.: PCT/US2019/019561

§ 371 Date: Aug. 18, 2020

Related U.S. Application Data
(60) Provisional application No. 62/635,417, filed on Feb. 26, 2018.

(51) Int. Cl. C12N 15/52; C12N 9/00; C12N 1/20

(52) U.S. Cl. CPC C12N 15/52; C12N 9/93; C12N 1/20; C12N 2500/36; C12N 2500/16; C12N 2500/34; C12Y 602/01001

(57) ABSTRACT

A method to improve the production of acetyl-CoA-derived biochemicals by overexpression of an acetyl-coenzyme A synthetase or acetate-CoA ligase from naturally acetate-utilizing organisms with or without an added acetate transporter. The production of free fatty acid and its derivatives from renewable carbon source was used as a non-limiting example. Using this approach, the production of free fatty acids with yield close to the maximum theoretical yield at high titer can be achieved. As such, this invention will provide the necessary framework to produce many other products sharing or branching out from the fatty acid synthesis pathway economically. These products include hydrocarbons, fatty alcohols, hydroxy fatty acids, dicarboxylic acids, fatty acid esters, etc.

### PRIOR RELATED APPLICATIONS

This application claims priority to U.S. Ser. No. 62/635,417, filed Feb. 26, 2018 and incorporated by reference in its entirety for all purposes.

### FEDERALLY SPONSORED RESEARCH STATEMENT

Not applicable.

### FIELD OF THE INVENTION

The invention relates to a method of increasing the production of acetyl-coA and products produced therefrom by using genetically engineered microorganisms. In more detail, the invention focuses on acetate utilization to improve acetyl-coA derived products.

### BACKGROUND OF THE INVENTION

As non-renewable feedstocks increase in price with reduced availability, and are associated with ecological hazards, there has been a movement towards using sustainable sources of feedstocks. Thus, there has been considerable effort towards producing feedstocks in bacteria, yeast and algae, as well as in using biological waste products, such as switch grass, corn husks, waste paper, and the like.

One of the biggest challenges in process scale-up of most biosynthesis systems is to achieve high product titer at high product yield and high product productivity. This is particularly for acetyl-CoA derived products where high concentration of acetate may be produced as a byproduct. The accumulation of acetate has shown to be detrimental to desired product formation.

Multiple approaches have been reported to reduce acetate accumulation. These include: 1) operate at a lower cultivating temperature; 2) use of fructose or glycerol as the carbon source; 3) divert the carbon overflow to a benign product; 4) use genetically engineered strains with deficiency in the acetate synthesis pathways. All of these approaches have their own advantages and disadvantages.

Therefore, there is still a need in the art for a biological system of producing Acetyl-coA derived products that is more efficient and cost effective than heretofore realized.

### SUMMARY OF THE INVENTION

In contrast to blocking acetate formation, as was done in some of the prior art approaches, we propose to re-utilize the acetate by converting it to acetyl-CoA. This conversion can be achieved by overexpressing the gene of acetyl-coenzyme A synthetase or acetate-CoA ligase from naturally acetate-utilizing organisms. Acetyl-coenzyme A synthetase or acetate-CoA ligase also function when there is a need to reabsorb the acetate formed when the strain grows at high rate.

Although an overexpressed native acetyl-coenzyme A synthetase has been studied in E. coli, the improvement in acetate utilization was not high because a large amount of acetate still formed at exponential growth stage (Lin et al., 2006).

Herein, we overexpress acetyl-coenzyme A synthetase or acetate-CoA ligase from natural acetate-utilizing organisms—e.g., microbes that are actively involved in acetate turnover, many (but not all) of which are methanogens, and most (if not all) of which are believed to belong to the Archebacteria.

We speculate that the acetyl-coenzyme A synthetase or acetate-CoA ligase from these unique organisms has evolved to have better enzymatic properties (such as Kcat and Km values) toward acetate utilization and therefore, their enzymes are a better choice for this purpose. Some suitable natural acetate-utilizing organisms and their ACS proteins are listed in the following table.

TABLE 1

| Organism | Acc. No. | Enzyme name |
|---|---|---|
| Methanosaeta thermophila PT | A0B8F1, | Acetyl-coenzyme |
| | ABK14976, | A synthetase |
| | ABK14977, | |
| | ABK15188 | |
| Methanosaeta harundinacea | KUK43842, | acetyl-coenzyme A |
| | KUK43843, | synthetase, |
| | KUK43844, | acetate--CoA ligase |
| | KUK45523, | |
| | KUK97549, | |
| | KUK97547, | |
| | WP_014586307, | |
| | WP_014586309, | |
| | WP_014586310 | |
| Methanosaeta concilii | WP_013718457, | acetate--CoA ligase |
| | WP_013718459, | |
| | WP_013718460, | |
| | WP_013720262, | |
| | WP_048131716, | |
| | P27095 | |
| Methanosaeta concilii GP6 | AEB67396 | Acetyl-coenzyme |
| | | A synthetase |
| Methanosaeta sp. ASM2 | OYV13150, | acetyl-CoA |
| | OYV13732, | synthetase |
| | OYV13862 | |
| Methanosaeta sp. NSP1 | OYV10542 | acetyl-coenzyme |
| | | A synthetase |
| Methanosaeta sp. NSM2 | OYV13002, | Acetyl-coenzyme |
| | OYV14207 | A synthetase |
| Methanobacteriaceae | KUK00397 | acetyl-coenzyme |
| archaeon 41_258 | | A synthetase |
| Methanothermobacter | BAW30830 | acetyl-coenzyme |
| sp. MT-2 | | A synthetase |
| Methanothermobacter | WP_048176124 | acetate--CoA ligase |
| sp. CaT2 | | |
| Methanothermobacter | WP_013295027 | acetate--CoA ligase |
| marburgensis | | |
| Methanothermobacter | ABC87078, | acetyl-CoA |
| thermautotrophicus | ABC87080 | synthetase 2 |
| Methanobacterium | WP_048080955, | acetate--CoA ligase |
| | WP_069584381 | |
| Methanobacterium | SCG85346, | Acetyl-coenzyme A |
| congolense | WP_071906527, | synthetase, |
| | WP_071907341 | acetate--CoA ligase |
| Methanobacterium | WP_081580334, | acetate--CoA ligase, |
| formicicum | WP_081944547, | acetyl-CoA |
| | WP_082055695, | synthetase AcsA2 |
| | AIS31518, | |
| | CEA13012 | |
| Methanobacterium formicicum | EKF85417 | acetate--CoA ligase |
| DSM 3637 | | |
| Methanobacterium lacus | WP_013644169 | acetate--CoA ligase |
| Methanobacterium paludis | WP_048188063, | acetate--CoA ligase |
| | AEG18848 | |
| Methanobacterium sp. | EKQ52488 | acetate--CoA ligase |
| Maddingley MBC34 | | |
| Methanobacterium sp. SMA-27 | WP_048191278 | acetate--CoA ligase |
| Methanobacterium sp. 42_16 | KUK73237 | acetate--CoA ligase |
| Archaeoglobus sulfaticallidus | WP_015590223 | acetate--CoA ligase |
| Archaeoglobus veneficus | WP_013683035 | acetate--CoA ligase |
| Archaeoglobus profundus | ADB57322 | acetate--CoA ligase |
| DSM 5631 | | |
| Archaeoglobus profundus | WP_048084686 | acetate--CoA ligase |
| Armatimonadetes bacterium | OYT69023 | acetate--CoA ligase |
| CP1_7O | | |
| Armatimonadetes | WP_072267913 | acetate--CoA ligase |
| bacterium DC | | |
| Armatimonadetes | WP_072260204 | acetate--CoA ligase |
| bacterium GBS | | |
| Armatimonadetes | WP_073995547 | acetate--CoA ligase |
| bacterium GXS | | |
| Armatimonadetes bacterium | OYT71558 | acetate--CoA ligase |
| JP3_11 | | |
| Calderihabitans maritimus | WP_088553916 | acetate--CoA ligase |
| Candidatus Altiarchaeales | OYT42298 | acetate--CoA ligase |
| archaeon ex4484_43 | | |
| Candidatus Schekmanbacteria | OGL40979 | acetate--CoA ligase |
| bacterium GWA2_38_9 | | |
| Desulfonauticus submarinus | WP_092065631 | acetate--CoA ligase |
| Desulfonatronospira | WP_008871338 | acetate--CoA ligase |
| thiodismutans | | |
| Desulfonatronum | WP_092120210 | acetate--CoA ligase |
| thiosulfatophilum | | |
| Desulfotomaculum | WP_031515925 | acetate--CoA ligase |
| alkaliphilum | | |
| Euryarchaeota archaeon | OYT57462 | acetate--CoA ligase |
| ex4484_162 | | |
| Nitrospirae bacterium | OGW42882 | acetate--CoA ligase |
| RBG_16_43_11 | | |
| Theionarchaea | KYK34138 | Acetyl-coenzyme |
| archaeon DG-70 | | A synthetase |
| Thermobaculum terrenum | WP_041424684 | acetate--CoA ligase |
| Thermobaculum terrenum | ACZ41798 | acetate--CoA ligase |
| ATCC BAA-798 | | |
| Thermodesulfobium narugense | WP_013756596 | acetate--CoA ligase |
| Thermoplasmatales archaeon | EMR73471 | acetyl-coenzyme |
| SCGC AB-539-N05 | | A synthetase |
| Thermoplasmatales | KYK27565 | acetyl-coenzyme |
| archaeon SGB-52-1 | | A synthetase |
| Thermoplasmatales | KYK21674 | acetyl-coenzyme |
| archaeon SGB-52-2 | | A synthetase |
| Thermoplasmatales | KYK34849 | acetyl-coenzyme |
| archaeon SGB-52-3 | | A synthetase |

The following table lists some acetyl-coenzyme A synthetase or acetate-CoA ligase with the identity more than 60 aligned with acetyl-coenzyme A synthetase (Acc. No. A0B8F1) from Methanosaeta thermophila exemplified herein. The acetyl-coenzyme A synthetase from Euryarchaeota archaeon ex4484_162 (Acc. No. OYT57462) shared an 60% identity with Acs1Mst.OYT57462.1 has the highest identity from non-Methanosaeta strains. The acetyl-coenzyme A synthetase from Methanothermobacter sp. MT-2 (Ace. No. BAW30830) shared an 58 identity with Acs1Mst. The acetyl-coenzyme A synthetase from Escherichia coli MG1655 (Acc. No. NP_418493) sharing a 45 identity is also listed below.

| Organism | Acc. No. | Identity |
|---|---|---|
| Methanosaeta thermophila PT | A0B8F1 | — |
| Methanosaeta concilii | WP_013720262 | 81% |
| Methanosaeta sp. NSM2 | OYV14207 | 81% |
| Methanosaeta concilii GP6 | AEB67396 | 81% |
| Methanosaeta concilii | WP_048131716 | 80% |
| Methanosaeta thermophila PT | ABK14976 | 80% |
| Methanosaeta thermophila PT | ABK14977 | 72% |
| Methanosaeta concilii | WP_013718459 | 72% |
| Methanosaeta harundinacea | KUK43843 | 70% |
| Methanosaeta harundinacea | KUK43842, | 69% |
| | KUK43844, | |
| | KUK97549, | |
| | KUK97547, | |
| | WP_014586307, | |
| | WP_014586309 | |
| Methanosaeta harundinacea | WP_014586310 | 69% |
| Methanosaeta thermophila PT | ABK15188 | 66% |
| Methanosaeta sp. ASM2 | OYV13862 | 66% |
| Methanosaeta concilii | WP_013718460, | 65% |
| | P27095 | |
| Methanosaeta sp. ASM2 | OYV13732 | 62% |
| Methanosaeta sp. NSM2 | OYV13002 | 62% |
| Methanosaeta sp. NSP1 | OYV10542 | 61% |
| Methanosaeta concilii | WP_013718457 | 60% |
| Euryarchaeota archaeon ex4484_162 | OYT57462 | 60% |
| Methanothermobacter sp. MT-2 | BAW30830 | 58% |
| Escherichia coli str. K-12 substr. MG1655 | NP_418493 | 45% |

From the work described herein, we have learned the following:

1) Acetyl-coA synthetase from natural acetate using organisms are more efficient than the E coli Acetyl-coA synthetase as these enzymes might evolved to have better enzymatic properties, such as Kcat and Km values, towards acetate.

2) Appropriate expression level of acetyl-coA synthetase is important. Our data showed that there is an optimal window, and that too little is inefficient, whilst too much will exert a metabolic burden on the cell.

3) Overexpression of acetyl-coA synthetase together with acetate transporter yields even better results than ACS alone.

4) The cultures become more robust (maintain productivity for a longer time) with overexpression of acetyl-coA synthetase, or acetyl-coA synthetase together with an acetate transporter.

5) Overexpression of acetyl-coA synthetase or acetyl-coA synthetase together with acetate transporter allows efficient utilization of acetate or acetate/sugar mixtures. This can help reduce the cost of scaling up the cultures.

In more detail, the invention is one or more of the following embodiments in any combination(s) thereof:

A recombinant microbe, said microbe overexpressing i) an acetyl-coenzyme A synthetase or an acetate-CoA ligase from a naturally acetate-utilizing organism, and optionally ii) an acetate transporter, said microbe having higher synthesis of acetyl-coA than a comparable microbe without i) or i) and ii).

Any microbe herein, said naturally acetate-utilizing organism selected from Methanosaeta thermophila PT, Methanosaeta harundinacea, Methanosaeta concilii, Methanosaeta sp. ASM2, Methanosaeta sp. NSP1, Methanosaeta sp. NSM2, Methanobacteriaceae archaeon 41_258, Methanothermobacter sp. MT-2, Methanothermobacter sp. CaT2, Methanothermobacter marburgensis, Methanothermobacter thermautotrophicus, Methanobacterium, Methanobacterium congolense, Methanobacterium formicicum, Methanobacterium formicicum DSM 3637, Methanobacterium lacus, Methanobacterium paludis, Methanobacterium sp. Maddingley MBC34, Methanobacterium sp. SMA-27, Methanobacterium sp. 42_16, Euryarchaeota archaeon ex4484_162 and Methanothermobacter sp. MT-2.

Any microbe herein, said acetyl-coenzyme A synthetase or an acetate-CoA ligase selected from those listed in Table 1.
Any microbe herein, said acetate transporter selected from ActP and SatP.
Any microbe herein, said acetyl-coenzyme A synthetase or said acetate-CoA ligase from Methanosaeta and said acetate transporter selected from ActP and SatP from Escherichia.
Any microbe herein, said microbe having a genotype of actP⁺⁺ and optionally acs1Mst⁺⁺ or acyl-ACP thioesterase⁺, actP⁺⁺, acs2Ea⁺⁺ or acyl-ACP thioesterase⁺, actP⁺⁺, acs3Mm⁺⁺.

In addition, any of the bacteria described herein can be combined with ΔfadD, although this deletion is not essential to the invention. Other host strain manipulations (such fadR, fabR, etc) can be used here to further improve fatty acid production, but they may not be relevant to the utilization of acetate. Thus, the background will vary with the desired product.
A recombinant microbe, said microbe having i) an inducible gene encoding a heterologous acetyl-coenzyme A synthetase or an acetate-CoA ligase from a naturally acetate-utilizing organism plus ii) an inducible gene encoding an acetate transporter, said microbe capable of producing at least 50% more of a product requiring acetyl-coenzyme A than a comparable microbe lacking i) and ii).
A recombinant microbe, said microbe having i) an overexpressed acetyl-coenzyme A synthetase or an acetate-CoA ligase from a naturally acetate-utilizing organism plus ii) overexpression of an acetate transporter, said naturally acetate-utilizing organism selected from Methanosaeta thermophila PT, Methanosaeta harundinacea, Methanosaeta sp. ASM2, Methanosaeta sp. NSP1, Methanosaeta sp. NSM2, Methanobacteriaceae archaeon 41_258, Methanothermobacter sp. MT-2, Methanothermobacter sp. CaT2, Methanothermobacter marburgensis, Methanothermobacter thermautotrophicus, Methanobacterium, Methanobacterium congolense, Methanobacterium formicicum, Methanobacterium formicicum DSM 3637, Methanobacterium lacus, Methanobacterium paludis, Methanobacterium sp. Maddingley MBC34, Methanobacterium sp. SMA-27, Methanobacterium sp. 42_16, Euryarchaeota archaeon ex4484_162 and Methanothermobacter sp. MT-2.
A recombinant microbe, said microbe having a genotype of i) acs1Mst⁺⁺ and optionally ii) actP⁺⁺ or having both i) and ii), or acyl-ACP thioesterase⁺, actP⁺⁺, acs2Ea⁺⁺ or acyl-ACP thioesterase⁺, actP⁺⁺, acs3Mm⁺⁺.
A recombinant microbe, said microbe being E. coli having a genotype of i) acs1Mst⁺⁺ and optionally ii) actP⁺⁺ or having both i) and ii), or acyl-ACP thioesterase⁺, actP⁺⁺, acs2Ea⁺⁺ or acyl-ACP thioesterase⁺, actP⁺⁺, acs3Mm⁺⁺.
A method of producing a product, comprising: inoculating a microbe as described herein into a nutrient broth; growing said microbe in said nutrient broth for a time sufficient to overexpress said i) and optionally ii) or both i) and ii) in an amount sufficient to make acetyl-coA and convert said acetyl-coA into an acetyl-co-A derived product, and isolating said acetyl-co-A derived product from said microbe, said nutrient broth, or both.
Any method herein, said nutrient broth supplemented with about 5-20 mg/ml? or about 10 mg/ml Mg.
Any method herein, further comprising feeding additional carbon source to said microbes when a pH of said nutrient broth becomes higher than 7.6.
Any method herein, wherein said acetyl-coA derived product is selected from free fatty acid, hydrocarbons, fatty alcohols, hydroxy fatty acids, dicarboxylic acids, and fatty acid esters.
Any method herein, said carbon source selected from glucose, sucrose, xylose, arabinose, galactose, mannose, acetate, glycerol, mixed sugars and hydrolysates with mixed sugars.
Any method herein, said acetyl-coenzyme A synthetase or acetate-CoA ligase and said acetate transporter being induced with about 100 μM of IPTG in said nutrient broth.
Any method herein, said nutrient broth comprising glucose, said acetyl-co-A derived product being free fatty acids, and said microbe producing 0.3 g of free fatty acid per gram of glucose.

Acyl-acyl carrier protein (ACP) thioesterase (herein known as “TE”) is an enzyme that terminates the intraplastidial fatty acid synthesis by hydrolyzing the acyl-ACP intermediates and releasing free fatty acids to be incorporated into glycerolipids. In plants, these enzymes are classified in two families, FatA and FatB, which differ in amino acid sequence and substrate specificity. Generally speaking, the N terminal (aa 1-98) of any acyl-ACP thioesterases controls the substrate specificity of the enzyme, and it is known how to change substrate specificity by swapping amino terminal domains.

Bacteria already have native acyl-ACP thioesterase proteins that can be used in the invention (e.g., FadM, TesA, TesB). These can be used as is, or up regulated or otherwise made to be overexpressed. However, any acyl-ACP thioesterase can also be added to the bacteria, and this is especially beneficial where one wants to generate a specific distribution of fatty acids, since the various enzymes have different substrate preferences, some producing longer fats and others short fats.

Many acyl-ACP thioesterase proteins are known and can be added to bacteria for use in the invention (e.g., CAA52070, YP_003274948, ACY23055, AAB71729, BAB33929, to name a few of the thousands of such proteins available), although we have used plasmids encoding plant genes herein. Such genes can be added by plasmid or other vector, or can be cloned directly into the genome. In certain species it may also be possible to genetically engineer the endogenous protein to by overexpressed by changing the regulatory sequences or removing repressors. However, overexpressing the gene by inclusion on selectable plasmids that exist in hundreds of copies in the cell may be preferred due to its simplicity, although permanent modifications to the genome may be preferred in the long term for genetic stability.

Other acyl ACP thioesterases include Umbellularia californica fatty acyl-ACP thioesterase (AAC49001), Cinnamomum camphora fatty acyl-ACP thioesterase (Q39473), Umbellularia californica fatty acyl-ACP thioesterase (Q41635), Myristica fragrans fatty acyl-ACP thioesterase (AAB71729), Myristica fragrans fatty acyl-ACP thioesterase (AAB71730), Elaeis guineensis fatty acyl-ACP thioesterase (ABD83939), Elaeis guineensis fatty acyl-ACP thioesterase (AAD42220), Populus tomentosa fatty acyl-ACP thioesterase (ABC47311), Arabidopsis thaliana fatty acyl-ACP thioesterase (NP_172327), Arabidopsis thaliana fatty acyl-ACP thioesterase (CAA85387), Arabidopsis thaliana fatty acyl-ACP thioesterase (CAA85388), Gossypium hirsutum fatty acyl-ACP thioesterase (Q9SQI3), Cuphea lanceolata fatty acyl-ACP thioesterase (CAA54060), Cuphea hookeriana fatty acyl-ACP thioesterase (AAC72882), Cuphea calophylla subsp. mesostemon fatty acyl-ACP thioesterase (ABB71581), Cuphea lanceolata fatty acyl-ACP thioesterase (CAC19933), Elaeis guineensis fatty acyl-ACP thioesterase (AAL15645), Cuphea hookeriana fatty acyl-ACP thioesterase (Q39513), Gossypium hirsutum fatty acyl-ACP thioesterase (AAD01982), Vitis vinifera fatty acyl-ACP thioesterase (CAN81819), Garcinia mangostana fatty acyl-ACP thioesterase (AAB51525), Brassica juncea fatty acyl-ACP thioesterase (ABI18986), Madhuca longifolia fatty acyl-ACP thioesterase (AAX51637), Brassica napus fatty acyl-ACP thioesterase (ABH11710), Oryza sativa (indica cultivar-group) fatty acyl-ACP thioesterase (EAY86877), Oryza sativa (japonica cultivar-group) fatty acyl-ACP thioesterase (NP-001068400), Oryza sativa (indica cultivar-group) fatty acyl-ACP thioesterase (EAY99617), and Cuphea hookeriana fatty acyl-ACP thioesterase (AAC49269).

In some embodiments, at least one acyl-ACP thioesterase gene is from a plant, for example overexpressed acyl-ACP thioesterase gene from Ricinus communis (XP_002515564.1), Jatropha curcas (ABU96744.1), Diploknema butyracea (AAX51636.1), Cuphea palustris (AAC49180.1), or Gossypium hirsutum (AAF02215.1 or AF076535.1), or an overexpressed hybrid acyl-ACP thioesterase comprising different thioesterase domains operably fused together (see WO2011116279, sequences expressly incorporated by reference herein). Preferably, the hybrid thioesterase includes an amino terminal region (˜aa 1-98 controls substrate specificity) of the acyl-ACP thioesterase from Ricinus communis or a 70, 80, 90 or 95% homolog thereto, or any TE with the desired substrate specificity, operably coupled to the remaining portion of the thioesterase from another species. In such manner, enzyme specificity can be tailored for the use in question.

As used herein, acetyl-CoA synthetase or acetate-CoA ligase are different names for the same enzyme (EC 6.2.1.1) involved in metabolism of acetate. It is in the ligase class of enzymes, meaning that it catalyzes the formation of a new chemical bond between two large molecules.

The two molecules joined together that make up Acetyl CoA synthetase are acetate and coenzyme A (CoA). The complete reaction with all the substrates and products included is:

ATP+Acetate+CoA<=>AMP+Pyrophosphate+Acetyl-CoA

As used herein, “enhanced amount” or “increased amount” means an improvement in production of acetyl coA, as compared to a comparable strain before it was engineered as described herein, e.g., lacking i) the added acetyl-coenzyme A synthetase or acetate-CoA ligase from a naturally acetate-utilizing organism, and ii) an acetate transporter. Since the A-coA is in flux and does not build up a substantial pool, we have measured A-coA increases by measuring an increase in fatty acid yield, determined by the ratio of grams of fatty acids produced to grams of glucose used. Obviously, in certain FAS mutants, it may not be possible to measure FA increase, and other proxies for Acetyl-coA could be used. Preferably, at least 25% improvement is observed, 30, 35, 40, 45, 50, 60, 70 or 80% improvement is observed.

As used herein, “fatty acids” means any saturated or unsaturated aliphatic acids having the common formulae of CₙH_{2n±x}COOH, wherein x≤n, which contains a single carboxyl group.

As used herein, “reduced activity” is defined herein to be at least a 75% reduction in protein activity, as compared with an appropriate control species. Preferably, at least 80, 85, 90, 95% reduction in activity is attained, and in the most preferred embodiment, the activity is eliminated (100%). Proteins can be inactivated with inhibitors, by mutation, or by suppression of expression or translation, by knock-out, by adding stop codons, by frame shift mutation, and the like.

By “knockout” or “null” mutant what is meant is that the mutation produces almost undetectable amounts of protein activity. A gene can be completely (100%) reduced by knockout or removal of part or all of the gene sequence. Use of a frame shift mutation, early stop codon, point mutations of critical residues, or deletions or insertions, and the like, can also completely inactivate (100%) gene product by completely preventing transcription and/or translation of active protein. All knockout mutants herein are signified by Agene where the gene name is identified above in Table A.

As used herein, “overexpression” or “overexpressed” is defined herein to be i) at least 150% of protein activity as compared with an appropriate control species, e.g., the same strain before the overexpression is engineered in, or ii) any activity in a species that otherwise wholly lacked the activity is considered overexpression. Preferably, the activity is increased 200-500%. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like. All overexpressed genes or proteins are signified herein by “⁺⁺”.

As used herein, all accession numbers are to GenBank unless indicated otherwise.

Exemplary gene or protein species are provided herein. However, gene and enzyme nomenclature varies widely, thus any protein (or gene encoding same) that catalyzes the same reaction can be substituted for a named protein herein. Further, while exemplary protein sequence accession numbers are provided herein, each is linked to the corresponding DNA sequence, and to related sequences. Further, related sequences can be identified easily by homology search and requisite activities confirmed as by enzyme assay, as is shown in the art.

E. coli gene and protein names (where they have been assigned) can be ascertained through ecoliwiki.net/ and enzymes can be searched through brenda-enzymes.info/. ecoliwiki.net/ in particular provides a list of alternate nomenclature for each enzyme/gene. Many similar databases are available including UNIPROTKB, PROSITE; 5 EC2PDB; ExplorEnz; PRIAM; KEGG Ligand; IUBMB Enzyme Nomenclature; IntEnz; MEDLINE; and MetaCyc, to name a few.

By convention, genes are written in italic, and corresponding proteins in regular font. E.g., fadD is the gene encoding FadD or acyl-CoA synthetase.

Generally speaking, we have used the gene name and protein names interchangeably herein, based on the protein name as provided in ecoliwiki.net. The use of a protein name as an overexpressed protein (e.g, FabH+) signifies that protein expression can occur in ways other than by adding a vector encoding same, since the protein can be upregulated in other ways. The use of FadD signifies that the protein can be downregulated in similar way, whereas the use of ΔfadD means that the gene has been directly downregulated.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations may be used:

| Abbreviation | Meaning |
|---|---|
| ACS | Acetyl coA synthetase |
| ActP | acetate transporter |
| EC | E. coli |
| FA | Fatty acid |
| FabZ | 3-hydroxyacyl-[acyl-carrier-protein] dehydratase FabZ |
| FadD | Long-chain-fatty-acid—CoA ligase |
| FadR | Fatty acid metabolism regulator protein |
| FAS | Fatty acid synthesis |
| FFA | Free fatty acids |
| MST | Methanosaeta thermophile |
| SucC | Succinate—CoA ligase [ADP-forming] subunit beta |
| TE | Thioesterase |
| Acc. No. | Accession Number |

### BRIEF DESCRIPTION OF THE DRAWINGS

FIG. 1. Fatty acid synthesis pathway.

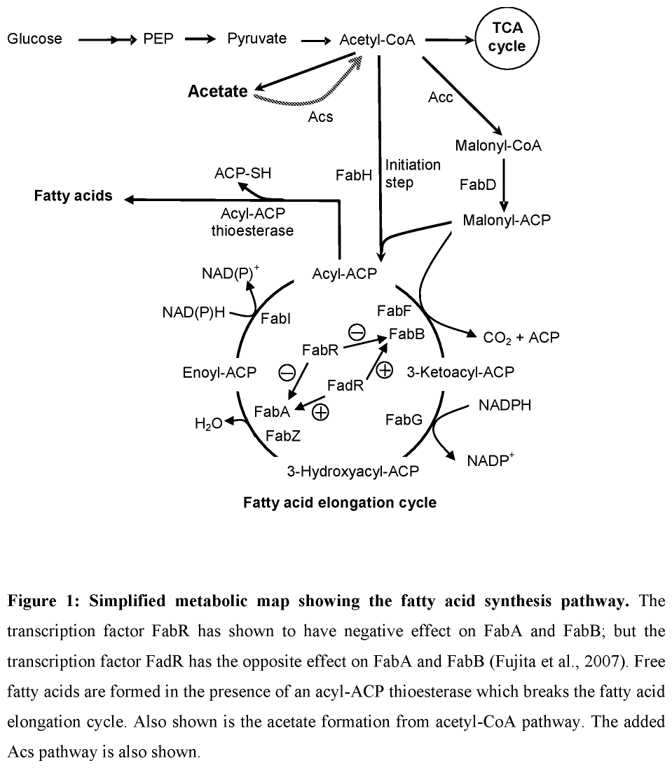

FIG. 2. Sequences.

### DESCRIPTION OF EMBODIMENTS OF THE INVENTION

We have shown that the acetyl-coenzyme A synthetase from M. thermophila performs much better than the native E. coli acetyl-coenzyme A synthetase using the production of fatty acid as an example. Furthermore, we have shown that co-expression of an acetate transporter further improves its performance, as measured by ratio of grams of fatty acids produced to grams of glucose used.

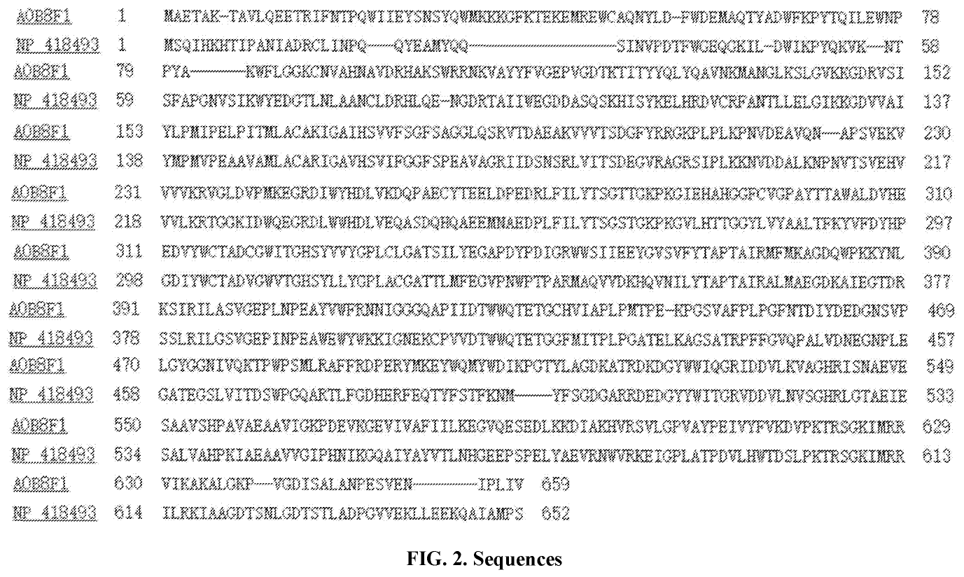

A comparison of the protein sequence between the M. thermophile Acs and E. coli Acs is shown in FIG. 2.

Strains table

| Strains | Genotype | Referance |
|---|---|---|
| ML103 | MG1655 ΔfadD | (Li et al., 2012)(Zhang et al., 2011) |
| ML212 | ML103 ΔsucC ΔfabR | (Li et al., 2017) |

Plasmids table

| Plasmids | Description | Referance |
|---|---|---|
| pWL1T | Modified pTrc99a carrying a long chain acyl-ACP thioesterase | (Li et al., 2017) |
| | (TE) under a constitutive promoter | |
| pWL1TZ | pWL1T overexpression of a β-hydroxyacyl-ACP dehydratase | (Li et al., 2017) |
| | (fabZ) under a constitutive promoter | |
| pWL1TZR | pWL1TZ overexpression of a transcriptional dual regulator | This invention |
| | (fadR) under a normal Ptrc promoter | |
| pWL8 | Replace araBAD promoter of pBAD33 using lad promoter | This invention |
| pWL8-acsEc | pWL8 carrying an acetyl-CoA synthetase (acs) from E. coli | This invention |
| pWL8-acs1Mst | pWL8 carrying a codon optimized acs from Methanosaeta strain | This invention |
| pWL8-actP-acsEc | pWL8-acsEc carrying an acetate transporter (actP) | This invention |
| pWL8-actP-acs1Mst | pWL8-acs1Mst carrying an actP | This invention |
| pWL8-actP-acs2Ea | Replace acs1Mst of pWL8-actP-acs1Mst using acs2Ea from | This invention |
| | Euryarchaeota archaeon ex4484_162 | |
| pWL8-actP-acs3Mm | Replace acs1Mst of pWL8-actP-acs1Mst using acs3Mm from | This invention |
| | Methanothermobacter sp. MT-2 | |

The metabolically engineered strains were studied in shake flasks as well as controlled bioreactor systems. For shake flask experiments, the strains were grown in 250 mL flasks, with 40 mL Luria-Bertani (LB) broth medium supplemented with glucose, appropriate IPTG, and 100 μg/mL ampicillin and/or 35 μg/mL chloramphenicol. The inoculation size is 1% (v/v). The cultures were grown in a rotary shaker at 250 rpm.

Samples of the media were taken at specific time after inoculation. The fatty acids were analyzed and quantified by GC/MS and GC/FID after extraction. Odd number saturated straight chain fatty acids, such as C13, C15 and/or C17 carbon chain length fatty acid, were used as the internal standard. The results shown in following tables are the sum of all major free fatty acids in the sample. The data shown are means for triplicate experiments.

Periodic-fed batch bioreactor experiments were performed on a 1-L bioreactor (BioFlo 110, New Brunswick Scientific Edison, N.J.) at 30° C. with 1% inoculation size in 600 mL LB broth supplied with glucose (and then fed at specific times), 100 μg/mL ampicillin and/or 35 μg/mL chloramphenicol, appropriate IPTG. The initial pH was adjusted to 7.3 with 2 N NaOH, the aeration was maintained at 1.0 vvm with filtered air. Continuous-fed bioreactor experiments were performed at the same conditions except using super broth as medium and continuous feeding glucose when pH in the medium was higher than 7.60.

### Overexpression of ACS or ACS and actP

Shake flask experiments were performed to demonstrate the effect of overexpression of acs1Mst and actP on the production of fatty acids as a non-limiting example. However, any other A-coA derived product could have been used for proof of concept.

The strain ML103 carrying the acyl-ACP thioesterase plasmid pWL1T produced moderate quantity of free fatty acids, about 1.52 g/L with a yield of 0.101 g/g on 15 g/L glucose. However, the strain ML103(pWL1T) bearing the acetyl-coenzyme A synthetase only produced a little fatty acid. When combining overexpression of acetate transporter ActP and acetyl-coenzyme A synthetase, the strains produced much higher fatty acid with more than 75% improvement compared with ML103(pWL1T+pWL8) on 15 g/L glucose. Furthermore, the corresponding improvement increased with the increase concentration of glucose. Moreover, the strains bearing an acetyl-coenzyme A synthetase from M. thermophila (Acs1Mst) always showed better performance than the strains bearing an acetyl-coenzyme A synthetase from E. coli (AcsEc).

| | | | | ~15 g/L glucose | | | ~30 g/L glucose | | | ~40 g/L glucose | | |
|---|---|---|---|---|---|---|---|---|---|---|---|---|
| | | | | Titer | Yield | Improvement^{#} | Titer | Yield | Improvement^{#} | Titer | Yield | Improvement^{#} |
| ML103 carrying | | | Relevant genotype | (g/L) | (g/g) | (%) | (g/L) | (g/g) | (%) | (g/L) | (g/g) | (%) |
| pWL1T | + | pWL8 | ΔfadD, acyl-ACP | 1.52 | 0.101 | — | 1.27 | 0.044 | — | 0.08 | 0.002 | — |
| | | | thioesterase⁺, cloning | | | | | | | | | |
| | | | vector pWL8 | | | | | | | | | |
| | | pWL8-acsEc | ΔfadD, acyl-ACP | 0.07 | 0.005 | −95.39 | 0.09 | 0.003 | −92.91 | 0.12 | 0.003 | 50.00 |
| | | | thioesterase⁺, acsEc⁺⁺ | | | | | | | | | |
| | | pWL8-acs1Mst | ΔfadD, acyl-ACP | 0.08 | 0.005 | −94.74 | 0.19 | 0.007 | −85.04 | 0.27 | 0.007 | 237.50 |
| | | | thioesterase⁺, | | | | | | | | | |
| | | | acs1Mst⁺⁺ | | | | | | | | | |
| | | pWL8-actP-acsEc | ΔfadD, acyl-ACP | 2.66 | 0.177 | 75.00 | 4.13 | 0.145 | 225.20 | 3.97 | 0.106 | 4862.50 |
| | | | thioesterase⁺, actP⁺⁺, | | | | | | | | | |
| | | | acsEc⁺⁺ | | | | | | | | | |
| | | pWL8-actP-acs1Mst | ΔfadD, acyl-ACP | 2.71 | 0.181 | 78.29 | 4.64 | 0.162 | 265.35 | 5.10 | 0.136 | 6275.00 |
| | | | thioesterase⁺, actP⁺⁺, | | | | | | | | | |
| | | | acs1Mst⁺⁺ | | | | | | | | | |
| | | pWL8-actP-acs2Ea | ΔfadD, acyl-ACP | 2.58 | 0.172 | 70.13 | — | — | — | — | — | — |
| | | | thioesterase⁺, actP⁺⁺, | | | | | | | | | |
| | | | acs2Ea⁺⁺ | | | | | | | | | |
| | | pWL8-actP-acs3Mm | ΔfadD, acyl-ACP | 2.59 | 0.173 | 70.51 | — | — | — | — | — | — |
| | | | thioesterase⁺, actP⁺⁺, | | | | | | | | | |
| | | | acs3Mm⁺⁺ | | | | | | | | | |

acsEc⁺⁺ = overexpression of E. coli Acs;
acs1Mst⁺⁺ = overexpression of M. thermophila Acs;
acs2Ea⁺⁺ = overexpression of E. archaeon Acs;
acs3Mm⁺⁺ = overexpression of Methanothermobacter sp. Acs;
actP⁺⁺ = overexpression of ActP;
^{#}Percentage improvement based on fatty acid produced by ML103(pWL1T + pWL8)

The strain ML103 carrying the acyl-ACP thioesterase and β-hydroxyacyl-ACP dehydratase (FabZ) plasmid pWL1TZ produced 3.28 g/L fatty acid on 15 g/L glucose, while strain ML103(pWL1TZ) bearing the acetyl-coenzyme A synthetase and acetate transporter ActP also got higher fatty acid under the same concentration of glucose. 7.73 g/L fatty acid was obtained from 40 g/L glucose when the strain carrying acetyl-coenzyme A synthetase Acs1Mst and acetate transporter ActP.

| | | | | ~15 g/L glucose | | | ~30 g/L glucose | | | ~40 g/L glucose | | |
|---|---|---|---|---|---|---|---|---|---|---|---|---|
| | | | | Titer | Yield | Improvement^{#} | Titer | Yield | Improvement^{#} | Titer | Yield | Improvement^{#} |
| ML103 carrying | | | Relevant genotype | (g/L) | (g/g) | (%) | (g/L) | (g/g) | (%) | (g/L) | (g/g) | (%) |
| pWL1TZ | + | pWL8 | ΔfadD, fabZ⁺⁺, acyl- | 3.28 | 0.219 | — | 4.23 | 0.141 | — | 0.22 | 0.005 | — |
| | | | ACP thioesterase⁺, | | | | | | | | | |
| | | | cloning vector pWL8 | | | | | | | | | |
| | | pWL8- | ΔfadD, fabZ⁺⁺, acyl- | 0.07 | 0.005 | −97.87 | 0.06 | 0.002 | −98.58 | 0.05 | 0.001 | −77.27 |
| | | acsEc | ACP thioesterase⁺, | | | | | | | | | |
| | | | acsEc⁺⁺ | | | | | | | | | |
| | | pWL8- | ΔfadD, fabZ⁺⁺, acyl- | 0.07 | 0.005 | −97.87 | 0.12 | 0.004 | −97.16 | 0.17 | 0.004 | −22.73 |
| | | acs1Mst | ACP thioesterase⁺, | | | | | | | | | |
| | | | acs1Mst⁺⁺ | 3.35 | 0.223 | 2.13 | 4.81 | 0.16 | 13.71 | 6.36 | 0.159 | 2790.91 |
| | | pWL8-actP- | ΔfadD, fabZ⁺⁺, acyl- | | | | | | | | | |
| | | acsEc | ACP thioesterase⁺, | | | | | | | | | |
| | | | actP⁺⁺, acsEc⁺⁺ | 3.6 | 0.24 | 9.76 | 5.7 | 0.19 | 34.75 | 7.73 | 0.193 | 3413.64 |
| | | pWL8-actP- | ΔfadD, fabZ⁺⁺, acyl- | | | | | | | | | |
| | | acs1Mst | ACP thioesterase⁺, | | | | | | | | | |
| | | | actP⁺⁺, acs1Mst⁺⁺ | | | | | | | | | |

acsEc⁺⁺ = overexpression of E. coli Acs;
acs1Mst⁺⁺ = overexpression of M. thermophila Acs;
actP⁺⁺ = overexpression of ActP;
fabZ⁺⁺ = overexpression of FabZ;
^{#}Percentage improvement based on fatty acid produced by ML103(pWL1TZ + pWL8)

The strain ML103 carrying the acyl-ACP thioesterase, FabZ and transcriptional dual regulator (FadR) plasmid pWL1TZR produced higher free fatty acids. However, the product yield decreases with increasing glucose concentrations. The strain with overexpression of E. coli acs and actP improved the fatty acid titer by 9.5% to 4.15 g/L while the strain with overexpression of M. thermophila acs and actP improved the fatty acid titer by more than 22% to 4.64 g/L with 15 g/L of glucose.

The strain with overexpression of M. thermophila acs only could produce 4.17 g/L fatty acid on 15 g/L glucose, a similar level that of the strain with overexpression of both E. coli acs and actP. Furthermore, the strain with overexpression of M. thermophila acs and actP maintained higher free fatty acid production with high yield even at higher glucose concentrations of 30 g/L and 40 g/L. This strain produced 12.04 g/L of free fatty acids with a yield of 0.301 g/g from 40 g/L of glucose. This high yield of 0.30 g/g is close to 90% of the maximum theoretical yield (e.g. the maximum theoretical yield of palmitic acid is 0.34 g/g glucose).

While the strains with overexpression of E. archaeon acs or Methanothermobacter sp.acs were shown to have lower the fatty acid titers than the strain with overexpression of M. thermophila acs at 15 g/L, 30 g/L and 40 g/L glucose, they did show a better improvement over the E. coli Acs at 15 g/L of glucose. These observations showed that Acs from different natural acetate using organisms can be used to improve acetate utilization but to various degrees.

| | | | | ~15 g/L glucose | | | ~30 g/L glucose | | | ~40 g/L glucose | | |
|---|---|---|---|---|---|---|---|---|---|---|---|---|
| | | | | Titer | Yield | Improvement^{#} | Titer | Yield | Improvement^{#} | Titer | Yield | Improvement^{#} |
| ML103 carrying | | | Relevant genotype | (g/L) | (g/g) | (%) | (g/L) | (g/g) | (%) | (g/L) | (g/g) | (%) |
| pWL1TZR | + | pWL8 | ΔfadD, fabZ⁺⁺, fadR⁺⁺, | 3.79 | 0.253 | — | 6.15 | 0.205 | — | 4.98 | 0.1245 | — |
| | | | acyl-ACP thioesterase⁺, | | | | | | | | | |
| | | | cloning vector pWL8 | | | | | | | | | |
| | | pWL8-acsEc | ΔfadD, fabZ⁺⁺, fadR⁺⁺, | 0.35 | 0.023 | −90.77 | 0.46 | 0.015 | −92.52 | 0.17 | 0.004 | −96.59 |
| | | | acyl-ACP thioesterase⁺, | | | | | | | | | |
| | | | acsEc⁺⁺ | | | | | | | | | |
| | | pWL8- | ΔfadD, fabZ⁺⁺, fadR⁺⁺, | 4.17 | 0.278 | 10.03 | 0.79 | 0.026 | −87.15 | 0.19 | 0.00475 | −96.18 |
| | | acs1Mst | acyl-ACP thioesterase⁺, | | | | | | | | | |
| | | | acs1Mst⁺⁺ | | | | | | | | | |
| | | pWL8-actP- | ΔfadD, fabZ⁺⁺, fadR⁺⁺, | 4.15 | 0.277 | 9.50 | 8.12 | 0.271 | 32.03 | 10.73 | 0.268 | 115.46 |
| | | acsEc | acyl-ACP thioesterase⁺, | | | | | | | | | |
| | | | actP⁺⁺, acsEC⁺⁺ | | | | | | | | | |
| | | pWL8-actP- | ΔfadD, fabZ⁺⁺, fadR⁺⁺, | 4.64 | 0.309 | 22.43 | 8.99 | 0.300 | 46.18 | 12.04 | 0.301 | 141.77 |
| | | acs1Mst | acyl-ACP thioesterase⁺, | | | | | | | | | |
| | | | actP⁺⁺, acs1Mst⁺⁺ | | | | | | | | | |
| | | pWL8-actP- | ΔfadD, fabZ⁺⁺, fadR⁺⁺, | 4.47 | 0.298 | 17.87 | 8.10 | 0.270 | 31.63 | 8.50 | 0.212 | 70.72 |
| | | acs2Ea | acyl-ACP thioesterase⁺, | | | | | | | | | |
| | | | actP⁺⁺, acs2Ea⁺⁺ | | | | | | | | | |
| | | pWL8-actP- | ΔfadD, fabZ⁺⁺, fadR⁺⁺, | 4.41 | 0.294 | 16.30 | 8.28 | 0.276 | 34.66 | 8.66 | 0.216 | 73.91 |
| | | acs3Mm | acyl-ACP thioesterase⁺, | | | | | | | | | |
| | | | actP⁺⁺, acs3Mm⁺⁺ | | | | | | | | | |

acsEc⁺⁺ = overexpression of E. coli Acs;
acs1Mst⁺⁺ = overexpression of M. thermophila Acs;
acs2Ea⁺⁺ = overexpression of E. archaeon Acs;
acs3Mm⁺⁺ = overexpression of Methanothermobacter sp. Acs;
actP⁺⁺ = overexpression of ActP;
fadR⁺⁺ = overexpression of FadR;
fabZ⁺⁺ = overexpression of FabZ;
^{#}Percentage improvement based on ML103(pWL1TZR + pWL8)

In summary, co-overexpression of M. thermophila acs and actP allows high fatty acid production with high yield. This strain can also maintain high titer and high yield even at high glucose concentrations.

### Time Profile of ACS, ACS, actP

Experiments were performed to track the time profiles of two strains and to compare their fatty acids production and the glucose utilization at 12 hour intervals. Although the glucose utilization time profiles were very similar among these two strains, the free fatty acids production were very different, with the strain carrying M. thermophila acs and E. coli actP producing the most fatty acids. Moreover, the production rate reached about 0.25 g/L/h between 12 and 24 hr even with these low cell density cultures with shake flask experiments.

| | Relevant | | Time (h) | | | | |
|---|---|---|---|---|---|---|---|
| Strains | geneotype | | 0 | 12 | 24 | 36 | 48 |
| ML103(pWL1TZR + | ΔfadD, fabZ⁺⁺, | Fatty acid (g/L) | 0 | 0.83 | 3.80 | 4.61 | 4.63 |
| pWL8-actP-acs1Mst) | fadR⁺⁺, | Residual | 15 | 8.57 | 1.71 | 0 | 0 |
| | acyl-ACP | glucose (g/L) | | | | | |
| | thioesterase⁺, | Acetate (g/L) | 0 | 0.23 | 0.05 | 0 | 0 |
| | acs1Mst⁺⁺, actP⁺⁺ | | | | | | |
| ML103(pWL1TZR + | ΔfadD, fabZ⁺⁺, | Fatty acid (g/L) | 0 | 1.45 | 3.30 | 3.80 | 3.79 |
| pWL8) | fadR⁺⁺, | Residual | 15 | 7.80 | 1.32 | 0 | 0 |
| | acyl-ACP | glucose (g/L) | | | | | |
| | thioesterase⁺, | Acetate (g/L) | 0 | 0.27 | 0.14 | 0.08 | 0.06 |
| | cloning vector | | | | | | |
| | pWL8 | | | | | | |

acs1Mst⁺⁺ = overexpression of M. thermophila Acs;
actP⁺⁺ = overexpression of ActP;
fadR⁺⁺ = overexpression of FadR;
fabZ⁺⁺ = overexpression of FabZ;

### Importance of ACS Expression Level

Experiments were performed to examine the expression level effect on strain performance. The control strain, ML103(pWL1TZ+pWL8), does not show any independent of the induction level. For this control strain, the fatty acid production is around 3.3 g/L. However, the acs1Mst⁺⁺, actP⁺⁺ carrying strain showed a strong dependence on the inducer, IPTG, concentrations. The performance dropped significantly at high induction level, probably due to over-burden by high levels of protein expression. The optimal for this construct is around 100 μM of IPTG.

| | | | IPTG (μM) | | | | | | | |
|---|---|---|---|---|---|---|---|---|---|---|
| Strains | Relevant Genotype | | 0 | 50 | 80 | 100 | 120 | 150 | 180 | 200 |
| ML103(pWL1TZ + | ΔfadD, fabZ⁺⁺, | Fatty acid (g/L) | 3.32 | 2.67 | 3.30 | 3.60 | 3.56 | 0.34 | 0.24 | 0.19 |
| pWL8-actP- | acyl-ACP | Residual glucose | 0 | 0 | 0 | 0 | 0 | 8.34 | 8.70 | 8.90 |
| acs1Mst) | thioesterase⁺, | (g/L) | | | | | | | | |
| | acs1Mst⁺⁺, actP⁺⁺ | Acetate (g/L) | 0 | 0 | 0 | 0 | 0 | 2.69 | 2.66 | 2.63 |
| ML103(pWL1TZ + | ΔfadD, fabZ⁺⁺, | Fatty acid (g/L) | 3.37 | 3.26 | 3.25 | 3.28 | 3.32 | 3.35 | 3.28 | 3.30 |
| pWL8) | acyl-ACP | Residual glucose | 0 | 0 | 0 | 0 | 0 | 0 | 0 | 0 |
| | thioesterase⁺, cloning | (g/L) | | | | | | | | |
| | vector pWL8 | Acetate (g/L) | 0 | 0 | 0 | 0 | 0 | 0 | 0 | 0 |

acs1Mst⁺⁺ = overexpression of M. thermophila Acs;
actP⁺⁺ = overexpression of ActP;
fabZ⁺⁺ = overexpression of FabZ.

In summary, an appropriate expression level of acetyl-coenzyme A synthetase and acetate transporter is needed for optimal strain performance. Too much expression strains the cell, and reduces production. We have found an optimal level to be about 80-120 μM or 100 μM IPTG to be effective, and titrations with other inducers can be performed as needed to determine optimal levels.

### Effect of Induction Timing

Experiments were performed to examine the effect of timing of induction of gene expression on strain performance. The control strain, ML103(pWL1TZ+pWL8), does not show any independent of the induction timing, as expected. For this control strain, the fatty acid production is around 3.3 g/L. However, the acs1Mst⁺⁺, actP⁺⁺ carrying strain showed little dependence on the induction time less than 3 hours after inoculation. However, the performance dropped significantly when the culture was induced at 5 hours after inoculation. These observations indicate that sufficient time should be allowed for cells to synthesize the enzyme(s) in order to have good performance.

| | | | | Induction time of 100 μM IPTG (h) | | | | | |
|---|---|---|---|---|---|---|---|---|---|
| Strains | Relevant phenotype | | no IPTG | 0 | 1 | 2 | 3 | 4 | 5 |
| ML103(pWL1TZ + | ΔfadD, fabZ⁺⁺, | Fatty acid (g/L) | 3.32 | 3.60 | 3.53 | 3.53 | 3.55 | 3.34 | 0.21 |
| pWL8-actP-acs1Mst) | acyl-ACP | | | | | | | | |
| | thioesterase⁺, | | | | | | | | |
| | acs1Mst⁺⁺, actP⁺⁺ | | | | | | | | |
| ML103(pWL1TZ + | ΔfadD, fabZ⁺⁺, | Fatty acid (g/L) | 3.37 | 3.28 | 3.35 | 3.36 | 3.30 | 3.29 | 3.34 |
| pWL8) | acyl-ACP | | | | | | | | |
| | thioesterase⁺, cloning | | | | | | | | |
| | vector pWL8 | | | | | | | | |

acs1Mst⁺⁺ = overexpression of M. thermophila Acs;
actP⁺⁺ = overexpression of ActP;
fabZ⁺⁺ = overexpression of FabZ;

In summary, induction at or near the beginning of the experiments (2-4 hrs) yields the best performance. This finding will facilitate future strain design and bioprocess operation as the results suggest it is possible to use constitutive promoter systems for the overexpression of M. thermophila Acs.

### Repeat Feed in Shake Flasks

Repeated feed experiments in shake flasks were performed to examine the ability of the strains to produce fatty acid in a sustained manner. In these experiments, 15 g/L glucose was added to the culture at every 24 h interval and the fatty acids in the culture were monitored. The control strain, ML103(pWL1TZ+pWL8), showed poor fatty acid production performance after the second sugar addition. However, the strain ML103(pWL1TZ+pWL8-actP-acs1Mst) obviously showed significant improvement on fatty acid production, glucose utilization and also acetate reduction. As such, this M. thermophila Acs carrying strain has potential for sustained fatty acid production.

| | | | Time (h) | | | | |
|---|---|---|---|---|---|---|---|
| strains | Relevant genotype | | 24 | 48 | 96 | 120 | 144 |
| ML103(pWL1TZ + | ΔfadD, fabZ⁺⁺, | Fatty acid (g/L) | 2.73 | 5.66 | 6.11 | 7.30 | 8.24 |
| pWL8-actP-acs1Mst) | acyl-ACP | Residual glucose (g/L) | 1.83 | 3.63 | 0.16 | 9.98 | 11.46 |
| | thioesterase⁺, | Acetate (g/L) | 0.04 | 0.03 | 0.00 | 1.00 | 1.03 |
| | acs1Mst⁺⁺, actP⁺⁺ | | | | | | |
| ML103(pWL1TZ + | ΔfadD, fabZ⁺⁺, | Fatty acid (g/L) | 2.39 | 4.17 | 5.21 | 5.27 | 5.99 |
| pWL8) | acyl-ACP | Residual glucose (g/L) | 2.18 | 6.88 | 14.90 | 28.18 | 53.08 |
| | thioesterase⁺, Cloning | Acetate (g/L) | 0.05 | 0.02 | 0.38 | 1.29 | 2.67 |
| | vector pWL8 | | | | | | |

acs1Mst⁺⁺ = overexpression of M. thermophila Acs;
actP⁺⁺ = overexpression of ActP;
fabZ⁺⁺ = overexpression of FabZ;

Previously we had shown the Mg⁺⁺ transporter or added MgCO₃ could improve the stability of fatty acid-producing strains (San et al., 2015). Here we examined repeated-batch with 10 g/L MgCO₃ supplement to raise the productivity. In these experiments, seed cultures were concentrated and re-suspended in LB to OD600 of around 20. After that, 15 g/L glucose was added at every 12 h interval. The strain ML103(pWL1TZ+pWL8-actP-acs1Mst) produced 14.72 g/L fatty acid after 4 batches or within 48 h, the yield and productivity of fatty acid reached to 0.245 g/g and 0.307 g/L/h, while the control strain only produced 7.41 g/L fatty acid with formation of 17.23 g/L acetate.

| | | | Time (h) | | | |
|---|---|---|---|---|---|---|
| strains | Relevant genotype | | 12 | 24 | 36 | 48 |
| ML103(pWL1TZ + | ΔfadD, fabZ⁺⁺, | Fatty acid (g/L) | 3.55 | 7.25 | 10.57 | 14.72 |
| pWL8-actP-acs1Mst) | acyl-ACP | Residual glucose | 0.12 | 0.07 | 0.07 | 0.02 |
| | thioesterase⁺, | (g/L) | | | | |
| | acs1Mst⁺⁺, actP⁺⁺ | Acetate (g/L) | 2.78 | 1.1 | 1.47 | 0 |
| ML103(pWL1TZ + | ΔfadD, fabZ⁺⁺, | Fatty acid (g/L) | 2.71 | 4.76 | 6.71 | 7.41 |
| pWL8) | acyl-ACP | Residual glucose | 0.03 | 0.04 | 0.03 | 0.26 |
| | thioesterase⁺, Cloning | (g/L) | | | | |
| | vector pWL8 | Acetate (g/L) | 3.46 | 3.95 | 7.87 | 17.23 |

acs1Mst⁺⁺ = overexpression of M. thermophila Acs;
actP⁺⁺ = overexpression of ActP;
fabZ⁺⁺ = overexpression of FabZ;

### Performance with Other Host Strains

Experiments were conducted to examine the fatty acid production by other strains carrying the M. thermophile acetyl-coenzyme A synthetase. The host strain ML212, which is a ΔsucC and ΔfabR derivative of the strain ML103, was used as an example. The data in the following table showed that the strain ML212 can improve fatty acid production equally well. Thus, the host strain is not critical.

| | | Fatty |
|---|---|---|
| Strains | Relevant Genotype | acid (g/L) |
| ML103(pWL1TZ+ | ΔfadD, fabZ⁺⁺, acyl-ACP thioesterase⁺, | 3.60 |
| pWL8-actP-acs1Mst)# | acs1Mst⁺⁺, actP⁺⁺ | |
| ML212 (pWL1TZ+ | ΔfadD, fabZ⁺⁺, acyl-ACP thioesterase⁺, | 3.84 |
| pWL8-actP-acs1Mst) | acs1Mst⁺⁺, actP⁺⁺ | |

acs1Mst⁺⁺ = overexpression of M. thermophila Acs;
actP⁺⁺ = overexpression of ActP;
fabZ⁺⁺ = overexpression of FabZ;
#Data from Experiment 3 above for comparison

### Periodic-Fed Batch Culture in Bioreactor

Experiments were performed on bioreactors to examine the periodic-fed batch fermentation ability. Strain carrying acs1Mst⁺⁺, actP⁺⁺ can maintain sustained fatty acid with additional batches of sugar to obtain higher fatty acid titer, yield and productivity than control strain. After 7 batches of 15 g/L glucose addition, 21.45 g/L of fatty acid was produced by strain carrying acs1Mst⁺⁺, actP⁺⁺. While the control strain, ML03(pWL1TZR+pWL8), only could feed 5 batches owing to slow utilization rate of glucose and produced 4.81 g/L fatty acid after 5 batches. These observations show that the strain carrying acs1Mst⁺⁺, actP⁺⁺ is more robust and can handle any adverse effect die to acetate accumulation (or prevent high level of acetate accumulation leading to any potential adverse effect for cell growth or product formation).

| | | | Glucose | Fatty acids | | | Maximum |
|---|---|---|---|---|---|---|---|
| | Relevant | Control | consumed | Titer | Yield | Productivity | acetate |
| strains | genotype | strategy | (g/L) | (g/L) | (g/g) | (g/L/h) | (g/L) |
| ML103(pWL1TZR + | ΔfadD, fabZ⁺⁺, | 10 g/L | 105 | 21.45 | 0.204 | 0.134 | 1.23 |
| pWL8-actP-acs1Mst) | fadR⁺⁺, acyl- | MgCO₃ add | | | | | |
| | ACP | at 21 h. | | | | | |
| | thioesterase⁺, | feed 15 g/L | | | | | |
| | acs1Mst⁺⁺, | glucose at | | | | | |
| | actP⁺⁺ | 21, 40, 60, | | | | | |
| | | 76, 90 and | | | | | |
| | | 112 h, total | | | | | |
| | | 7 batches. | | | | | |
| ML103(pWL1TZR + | ΔfadD, fabZ⁺⁺, | 10 g/L | 75 | 4.81 | 0.064 | 0.030 | 2.26 |
| pWL8) | fadR⁺⁺, acyl- | MgCO₃ add | | | | | |
| | ACP | at 21 h. | | | | | |
| | thioesterase⁺, | feed 15 g/L | | | | | |
| | cloning vector | glucose at | | | | | |
| | pWL8 | 21, 40, 60 | | | | | |
| | | and 76 h, | | | | | |
| | | total 5 | | | | | |
| | | batches. | | | | | |

acs1Mst⁺⁺ = overexpression of M. thermophila Acs;
actP⁺⁺ = overexpression of ActP;
fadR⁺⁺ = overexpression of FadR;
fabZ⁺⁺ = overexpression of FabZ;

### Continuous-Fed Culture

Experiments were performed on bioreactors to examine the continuous-fed fermentation ability. The strain carrying acs1Mst⁺⁺, actP⁺⁺ produced 56.48 g/L fatty acids after 65 h when super broth was used as the starting medium, with continuous feeding of glucose when pH in the medium reached higher than 7.60, and a recycling container to recycle trapped cells was used. The corresponding yield and overall production rate were 0.339 g/g and 0.869 g/L/h, respectively. The fatty acid productivity reached to a high rate of 1.177 g of fatty acids/L/h during the production phase. To our best knowledge, these are the highest reported values for fatty acid titer, yield and productivity by an E. coli culture.

| | | | Glucose | Fatty acids | | | Maximum |
|---|---|---|---|---|---|---|---|
| | Relevant | Control | consumed | Titer | Yield | Productivity | acetate |
| strains | genotype | strategy | (g/L) | (g/L) | (g/g) | (g/L/h) | (g/L) |
| ML103(pWL1TZR + | ΔfadD, fabZ⁺⁺, | 10 g/L | 167 | 56.48 | 0.339 | 0.869 | 0.60 |
| pWL8-actP-acs1Mst) | fadR⁺⁺, acyl- | MgCO₃ add | | | | | |
| | ACP | at 17 h. | | | | | |
| | thioesterase⁺, | continuous | | | | | |
| | acs1Mst⁺⁺, | feed 500 | | | | | |
| | actP⁺⁺ | g/L glucose | | | | | |
| | | using pH as | | | | | |
| | | a indictor | | | | | |
| | | (pH ≥ 7.60, | | | | | |
| | | start feed) | | | | | |

acs1Mst⁺⁺ = overexpression of M. thermophila Acs;
actP⁺⁺ = overexpression of ActP;
fadR⁺⁺ = overexpression of FadR;
fabZ⁺⁺ = overexpression of FabZ;

### Various Carbon Sources

Experiments were performed to examine repeated batch fermentation using acetate as the sole carbon source. The seed cultures were concentrated and re-suspended in LB to OD600 of around 20, 10 g/L MgCO₃ was added at the beginning, and 5 g/L acetate was fed at every 12 h interval. The strain carrying acs1Mst⁺⁺, actP⁺⁺ produced 3.45 g/L fatty acids after 4 batches or 48 h while the control strain just produced 2.77 g/L fatty acid with 8.44 g/L acetate unused.

| | | | Time (h) | | | |
|---|---|---|---|---|---|---|
| strains | Relevant genotype | | 12 | 24 | 36 | 48 |
| ML103(pWL1TZ + | ΔfadD, fabZ⁺⁺, acyl- | Fatty acid (g/L) | 1.71 | 2.49 | 3.04 | 3.45 |
| pWL8-actP-acs1Mst) | ACP thioesterase⁺, | Residual acetate (g/L) | 0 | 0.52 | 0.71 | 1.05 |
| | acs1Mst⁺⁺, actP⁺⁺ | | | | | |
| ML103(pWL1TZ + | ΔfadD, fabZ⁺⁺, acyl- | Fatty acid (g/L) | 1.37 | 2.13 | 2.67 | 2.77 |
| pWL8) | ACP thioesterase⁺, | Residual acetate (g/L) | 0.04 | 1.02 | 4.55 | 8.44 |
| | Cloning vector pWL8 | | | | | |

acs1Mst⁺⁺ = overexpression of M. thermophila Acs;
actP⁺⁺ = overexpression of ActP;
fabZ⁺⁺ = overexpression of FabZ;

Experiments were performed to examine the repeated batch fermentation using acetate mixed with glucose or glycerol as the carbon source. The seed cultures were concentrated and re-suspended in LB to OD600 of around 20, 10 g/L MgCO₃ was added at the beginning, and some amount of acetate mixed with glucose or glycerol was fed at every 12 h interval. After 3 batches or 36 h, the strain carrying acs1Mst⁺⁺, actP⁺⁺ produced 14.06 g/L fatty acids with a yield of 0.234 g/g when 15 g/L glucose and 5 g/L acetate was used as the carbon source. Feeding with 7.5 g/L glucose and 5 g/L acetate could improve the yield to 0.271 g/g. Moreover, the yield could be further improved when feeding carbon source containing glycerol, and reached to more than 0.27 g/g from 3 batches of 15 g/L glycerol and 0-4 g/L acetate.

| | | | Fatty acid | |
|---|---|---|---|---|
| | | | Titer | Yield |
| Strains | Relevant genotype | Feeding carbon source | (g/L) | (g/g) |
| ML103(pWL1TZ+ | ΔfadD, fabZ⁺⁺, acyl- | 3 batches × (15 g/L glucose) | 6.71 | 0.149 |
| pWL8) | ACP thioesterase+, | | | |
| # | Cloning vector | | | |
| | pWL8 | | | |
| ML103(pWL1TZ+ | ΔfadD, fabZ⁺⁺, acyl- | 3 batches × (15 g/L glucose) | 10.57 | 0.235 |
| pWL8- | ACP thioesterase⁺ | | | |
| actP-acs1Mst) # | acs1Mst⁺⁺, actP⁺⁺ | | | |
| ML103(pWL1TZ+ | ΔfadD, fabZ⁺⁺, acyl- | 3 batches × (15 g/L glucose | 14.06 | 0.234 |
| pWL8- | ACP thioesterase⁺ | +5 g/L acetate) | | |
| actP-acs1Mst) | acs1Mst⁺⁺, actP⁺⁺ | | | |
| ML103(pWL1TZ+ | ΔfadD, fabZ⁺⁺, acyl- | 3 batches × (7.5 g/L glucose | 10.17 | 0.271 |
| pWL8- | ACP thioesterase⁺ | +5 g/L acetate) | | |
| actP-acs1Mst) | acs1Mst⁺⁺, actP⁺⁺ | | | |
| ML103(pWL1TZ+ | ΔfadD, fabZ⁺⁺, acyl- | 3 batches × (15 g/L glycerol) | 12.45 | 0.277 |
| pWL8- | ACP thioesterase⁺ | | | |
| actP-acs1Mst) | acs1Mst⁺⁺, actP⁺⁺ | | | |
| ML103(pWL1TZ+ | ΔfadD, fabZ⁺⁺, acyl- | 3 batches × (15 g/L glycerol + | 14.41 | 0.283 |
| pWL8- | ACP thioesterase⁺ | 2 g/L acetate) | | |
| actP-acs1Mst) | acs1Mst⁺⁺, actP⁺⁺ | | | |
| ML103(pWL1TZ+ | ΔfadD, fabZ⁺⁺, acyl- | 3 batches × (15 g/L glycerol + | 15.00 | 0.278 |
| pWL8- | ACP thioesterase⁺ | 3 g/L acetate) | | |
| actP-acs1Mst) | acs1Mst⁺⁺, actP⁺⁺ | | | |
| ML103(pWL1TZ+ | ΔfadD, fabZ⁺⁺, acyl- | 3 batches × (15 g/L glycerol + | 15.48 | 0.272 |
| pWL8- | ACP thioesterase⁺ | 4 g/L acetate) | | |
| actP-acs1Mst) | acs1Mst⁺⁺, actP⁺⁺ | | | |

acs1Mst⁺⁺ = overexpression of M. thermophila Acs;
actP⁺⁺ = overexpression of ActP;
fabZ⁺⁺ = overexpression of FabZ;
# Data from Experiment 5b above for comparison

Experiments were performed to examine fermentation on mix sugar using 7.5 g/L glucose and 7.5 g/L galactose. After 48 h, the strain carrying acs1Mst⁺⁺, actP⁺⁺ produced 4.22 g/L fatty acid while control strain just produced 3.38 g/L fatty acid.

| | | Fatty acid |
|---|---|---|
| Strains | Relevant Genotype | (g/L) |
| ML103(pWL1TZR+ | ΔfadD, fabZ++, fadR⁺⁺, acyl-ACP | 4.22 |
| pWL8-actP-acs1Mst) | thioesterase+, acs1Mst++, actP++ | |
| ML103(pWL1TZR+ | ΔfadD, fabZ++, fadR⁺⁺, acyl-ACP | 3.38 |
| pWL8) | thioesterase+, cloning vector pWL8 | |

acs1Mst++ = overexpression of M. thermophila Acs;
actP++ = overexpression of ActP; fadR++ = overexpression of FadR;
fabZ++ = overexpression of FabZ;

In summary, these results showed the strain carrying acs1Mst⁺⁺, actP⁺⁺ could ferment efficiently various carbon sources such as sugar, acetate, glycerol and their mixture, and even could use acetate as sole carbon source. Further, with optimized culture conditions, yields of 0.177 g of fatty acids/L/h during the production phase.

The following publications are incorporated by reference in their entirety for all purposes herein.
US20040199941 INCREASED BACTERIAL COA AND ACETYL-COA POOLS
US20100248233 ACETYL-COA PRODUCING ENZYMES IN YEAST
US20170159075 MICROORGANISMS AND METHODS FOR IMPROVING PRODUCT YIELDS ON METHANOL USING ACETYL-COA SYNTHESIS
U.S. Pat. No. 9,598,696 BACTERIA AND METHOD FOR SYNTHESIZING FATTY ACIDS
WO2017189421 REDOX BALANCING IN YEAST.
Aristidou, A A, San, K. Y., Bennett, G. N., 1995. Metabolic engineering of Escherichia coli to enhance recombinant protein production through acetate reduction. Biotechnol. Prog. 11, 475-478. doi:10.1021/bp00034a019
Chang, D., Shin, S., Rhee, J., 1999. Acetate Metabolism in a pta Mutant of Escherichia coli W3110: Importance of Maintaining Acetyl Coenzyme A Flux for Growth and Survival Acetate Metabolism in a pta Mutant of Escherichia coli W3110: Importance of Maintaining Acetyl Coenzyme A Flux for Gro. J. Bacteriol. 181, 6656-6663.
Eiteman, M. A., Altman, E., 2006. Overcoming acetate in Escherichia coli recombinant protein fermentations. Trends Biotechnol. doi:10.1016/j.tibtech.2006.09.001
Farmer, W. R., Liao, J. C., 1997. Reduction of aerobic acetate production by Escherichia coli. Appl. Environ. Microbiol. 63, 3205-3210.
Fujita, Y., Matsuoka, H., Hirooka, K., 2007. Regulation of fatty acid metabolism in bacteria. Mol. Microbiol. doi:10.1111/j.1365-2958.2007.05947.x
Lee, S. Y., 1996. High cell-density culture of Escherichia coli. Trends Biotechnol. 14, 98-105. doi:10.1016/0167-7799(96)80930-9
Li, M., Zhang, X., Agrawal, A., San, K. Y., 2012. Effect of acetate formation pathway and long chain fatty acid CoA-ligase on the free fatty acid production in E. coli expressing acy-ACP thioesterase from Ricinus communis. Metab. Eng. 14, 380-387. doi:10.1016/j.ymben.2012.03.007
Li, W., Wu, H., Li, M., San, K.-Y., 2017. Effect of NADPH availability on free fatty acid production in E. coli. Biotechnol. Bioeng. doi:10.1002/bit.26464
Lin, H., Castro, N. M., Bennett, G. N., San, K.-Y. Y., 2006. Acetyl-CoA synthetase overexpression in Escherichia coli demonstrates more efficient acetate assimilation and lower acetate accumulation: A potential tool in metabolic engineering. Appl. Microbiol. Biotechnol. 71, 870-874. doi:10.1007/s00253-005-0230-4
Luo, Q., Shen, Y.-L., Wei, D.-Z., Cao, W., 2006. Optimization of culture on the overproduction of TRAIL in high-cell-density culture by recombinant Escherichia coli. Appl. Microbiol. Biotechnol. 71, 184-191. doi:10.1007/s00253-005-0131-6
San, K.-Y., Han, S., Li, W., Li, M., Li, Z., 2015. Fatty Acids With Mg Transporter and Mg.
THIEL, et al., The Effect Of Enhanced Acetate Influx On Synechocystis Sp. Pcc 6803 Metabolism, 16:21 (2017).
VEERAVALLI, et al., Strain Engineering To Reduce Acetate Accumulation During Microaerobic Growth Conditions In Escherichia coli, Biotechnol Prog (2017).
WOLFE, The Acetate Switch. Microbiol Mol Bio Rev, 69:12-50 (2005).
Zhang, X., Li, M., Agrawal, A., San, K.-Y., 2011. Efficient free fatty acid production in Escherichia coli using plant acyl-ACP thioesterases. Metab. Eng. 13, 713-722. doi:10.1016/j.ymben.2011.09.007

1) A recombinant microbe, said microbe overexpressing i) an acetyl-coenzyme A synthetase or an acetate-CoA ligase from a natural acetate-utilizing organism, and ii) an acetate transporter, said microbe having a higher rate of acetyl-coA synthesis than a comparable microbe without both i and ii.

2) The recombinant microbe of claim 1, said naturally acetate-utilizing organism selected from Methanosaeta thermophila PT, Methanosaeta harundinacea, Methanosaeta concilii, Methanosaeta sp. ASM2, Methanosaeta sp. NSP1, Methanosaeta sp. NSM2, Methanobacteriaceae archaeon 41_258, Methanothermobacter sp. MT-2, Methanothermobacter sp. CaT2, Methanothermobacter marburgensis, Methanothermobacter thermautotrophicus, Methanobacterium, Methanobacterium congolense, Methanobacterium formicicum, Methanobacterium formicicum DSM 3637, Methanobacterium lacus, Methanobacterium paludis, Methanobacterium sp. Maddingley MBC34, Methanobacterium sp. SMA-27, and Methanobacterium sp. 42_16.

3) The recombinant microbe of claim 1, wherein said acetyl-coenzyme A synthetase or acetate-CoA ligase is selected from those listed in Table 1.

4) The recombinant microbe of claim 1, wherein said acetate transporter is selected from ActP and SatP.

5) The recombinant microbe of claim 1, wherein said acetyl-coenzyme A synthetase or said acetate-CoA ligase is from Methanosaeta and said acetate transporter is selected from ActP and SatP from Escherichia.

6) The recombinant microbe of claim 1, said microbe having a genotype of i) acs1Mst⁺⁺ or acs2Ea⁺⁺ or acs3Mm⁺⁺ and ii) actP⁺⁺.

7) The recombinant microbe of claim 1, said microbe capable of producing at least 50% more of a product requiring acetyl-coenzyme A than a comparable microbe lacking i) and ii).

8) The recombinant microbe of claim 7, wherein said naturally acetate-utilizing organism is selected from Methanosaeta thermophila PT, Methanosaeta harundinacea, Methanosaeta sp. ASM2, Methanosaeta sp. NSP1, Methanosaeta sp. NSM2, Methanobacteriaceae archaeon 41_258, Methanothermobacter sp. MT-2, Methanothermobacter sp. CaT2, Methanothermobacter marburgensis, Methanothermobacter thermautotrophicus, Methanobacterium, Methanobacterium congolense, Methanobacterium formicicum, Methanobacterium formicicum DSM 3637, Methanobacterium lacus, Methanobacterium paludis, Methanobacterium sp. Maddingley MBC34, Methanobacterium sp. SMA-27, and Methanobacterium sp. 42_16.

9) A recombinant microbe, said microbe having i) an overexpressed acetyl-coenzyme A synthetase or an acetate-CoA ligase from a naturally acetate-utilizing organism, plus optionally ii) overexpression of an acetate transporter, said naturally acetate-utilizing organism selected from Methanosaeta thermophila PT, Methanosaeta harundinacea, Methanosaeta sp. ASM2, Methanosaeta sp. NSP1, Methanosaeta sp. NSM2, Methanobacteriaceae archaeon 41_258, Methanothermobacter sp. MT-2, Methanothermobacter sp. CaT2, Methanothermobacter marburgensis, Methanothermobacter thermautotrophicus, Methanobacterium, Methanobacterium congolense, Methanobacterium formicicum, Methanobacterium formicicum DSM 3637, Methanobacterium lacus, Methanobacterium paludis, Methanobacterium sp. Maddingley MBC34, Methanobacterium sp. SMA-27, and Methanobacterium sp. 4216.

10) (canceled)

11) A recombinant microbe, said microbe being E. coli having a genotype of i) acs1Mst⁺⁺ or acs2Ea⁺⁺ or acs3Mm⁺⁺ and ii) actP⁺⁺ and optionally iii) acyl-ACP thioesterase⁺.

12) A method of producing a product, comprising:
a) inoculating a microbe of claim 1 into a nutrient broth containing a carbon source;
b) growing said microbe in said nutrient broth for a time sufficient to overexpress said i) and ii)(if present) in an amount sufficient to make acetyl-coA and convert said acetyl-coA into an acetyl-co-A derived product, and
c) isolating said acetyl-co-A derived product from said microbe, said nutrient broth, or both.

13) The method of claim 12, said nutrient broth supplemented with about 10 mg Mg.

14) The method of claim 12, further comprising feeding additional carbon source to said microbes when a pH of said nutrient broth becomes higher than 7.6.

15) The method of claim 12, wherein said acetyl-coA derived product is selected from free fatty acid, hydrocarbons, fatty alcohols, hydroxy fatty acids, dicarboxylic acids, and fatty acid esters.

16) The method of claim 12, wherein said carbon source is selected from glucose, sucrose, xylose, arabinose, galactose, mannose, acetate, glycerol, sugar mixtures, and hydrolysate with mixed sugars.

17) The method of claim 12, wherein said carbon source is acetate.

18) The method of claim 12, said nutrient broth comprising glucose, said acetyl-co-A derived product being free fatty acids, and said microbe producing 0.3 g of free fatty acid per gram of glucose.

19) The method of claim 12, wherein an induction level of said i) and ii) expression is optimized to not be so high as to slow cell growth by >10% or so low as to produce <90% of a theoretical maximum of product.

20) The method of claim 12, said acetyl-coenzyme A synthetase or acetate-CoA ligase and said acetate transporter being inducible with about 100 μM of IPTG in said nutrient broth.

21) A method of producing a product, comprising:
a) inoculating a microbe of claim 9 into a nutrient broth containing a carbon source;
b) growing said microbe in said nutrient broth for a time sufficient to overexpress said i) and ii) (if present) in an amount sufficient to make acetyl-coA and convert said acetyl-coA into an acetyl-co-A derived product, and
c) isolating said acetyl-co-A derived product from said microbe, said nutrient broth, or both.
